(19) United States
(12) Patent Application Publication
(10) Pub. No.: US 2014/0221986 A1
(43) Pub. Date: Aug. 7, 2014

(54) MULTI-FUNCTIONAL MEDICAL DEVICE AND RELATED METHODS OF USE

(71) Applicant: Boston Scientific Scimed, Inc., Maple Grove, MN (US)

(72) Inventor: Gary KAPPEL, Acton, MA (US)

(73) Assignee: Boston Scientific Scimed, Inc., Maple Grove, MN (US)

(21) Appl. No.: 14/172,647

(22) Filed: Feb. 4, 2014

Related U.S. Application Data
(60) Provisional application No. 61/760,919, filed on Feb. 5, 2013.

(51) Int. Cl. A61B 17/00

(52) U.S. Cl. CPC A61B 17/00234
USPC 606/1

(57) ABSTRACT

A medical device may include an elongate member having a proximal end, a distal end, and a lumen extending therebetween. In addition an end-effector may be disposed at the distal end of the elongate member, the end-effector may include a first arm and a second arm. Additionally, the first arm may define a first slot and a second slot. Further, the medical device may include an actuating member, the actuating member may include a plurality pins, and wherein each of the pins may slideably engage the first and second slots.

### CROSS-REFERENCE TO RELATED APPLICATIONS

This application claims the benefit of priority from U.S. Provisional Application No. 61/760,919, filed on Feb. 5, 2013, the entirety of which is incorporated by reference herein.

### FIELD

Embodiments of this disclosure relate generally to medical devices and procedures. In particular, embodiments of the present disclosure relate to minimally invasive medical devices and procedures for manipulating tissues from inside a patient's body.

### BACKGROUND

An endoscopic procedure typically involves indirect observation of a surgical field through an endoscope or similar device inserted through an incision or a natural anatomical opening. The endoscope generally takes the form of a long, flexible tube, with one or more channels for inserting medical devices. Endoscopes provide platforms for employing numerous tools as end-effectors, such as devices to grasp, clip, sever, and remove objects from inside the body.

A pivoting end-effector typically includes at least two or more arms connected by at least a fulcrum, of which one or more of the arms may rotate with respect to at least another arm, and a mechanism to couple a force to one or more arms to pivot the arms with respect to each other. The location at which a force is applied to the arms affects the performance parameters (e.g., torque and angular speed) of the end-effector. In addition, the manner in which a force is distributed along the arm may have an impact on the durability, size, and/or load bearing capacity of the end-effector. Thus, it is desired to apply a force to an end-effector arm at multiple contacting locations and to vary the torque and angular speed profile for the end-effector.

### SUMMARY OF THE DISCLOSURE

In one embodiment, a medical device may include an elongate member having a proximal end, a distal end, and a lumen extending therebetween. The medical device also may include an end-effector disposed at the distal end of the elongate member, the end-effector may include a first arm and a second arm. Additionally, the first arm may define a first slot and a second slot. Further, the medical device may include an actuating member, the actuating member may include a plurality pins, and wherein each of the pins may slideably engage the first and second slots.

In various embodiments, the medical device may include one or more of the following additional features: the first slot is proximal to the second slot; at least one of the first and second slots defines a curvilinear path; at least one of the first and second slots defines a linear path; at least one of the plurality of pins is selectively engageable; at least one of the plurality of pins includes a deformable material; the plurality of pins includes a first pin and a second pin; the first and second pins are comprised of different materials; and the second arm is fixed with respect to the elongate member.

In another embodiment, a medical device may include an elongate member having a proximal end, a distal end, and a lumen extending therebetween. Additionally, the medical device may include an end-effector disposed at the distal end of the elongate member, the end-effector may include a first arm and a second arm. The medical device also may include an actuating member, the actuating member may include a plurality of pins; wherein each of the pins may be configured to transfer an applied force to the first arm; and wherein the first arm may be configured to rotate with respect to the second arm upon receiving the applied force.

In various embodiments, a medical device may include one or more of the following additional features: the first arm defines a first slot and a second slot, the first slot is located distally to the second slot; at least one of the first and second slots defines a curvilinear path; at least one of the plurality of pins is selectively engageable; at least one of the plurality of pins includes a deformable material; and the second arm is fixed with respect to the elongate member.

Further, in another embodiment, a medical device may include an elongate member having a proximal end, a distal end, and a lumen extending therebetween. In addition, the medical device may include an end-effector disposed at the distal end of the elongate member, the end-effector may include a first arm and a second arm. The first arm may define a first slot and a second slot, the first slot and the second slot may have a different profile from each other. In addition, the medical device may include an actuating member configured to transfer a plurality of applied forces to the first and second slots of the first arm; and wherein the first arm may be configured to rotate with respect to the second arm upon receiving the applied force.

In various embodiments, the medical device may include one or more of the following additional features: at least one of the first and second slots define a curvilinear path; at least one of the first and second slots define a linear path; the actuating member is configured to transfer the plurality of applied forces to the first slot; the plurality of applied forces including a first force and a second force; the first force is configured to be applied to the first slot and the second force is configured to be applied to the second slot; and the first force is different from the second force.

Additional objects and advantages of the present disclosure will be set forth in part in the description, which follows, and in part will be obvious from the description, or may be learned by practice of the present disclosure. The objects and advantages of the present disclosure will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate embodiments of the disclosure and together with the description, serve to explain the principles of the disclosure. Wherever possible, the same reference numbers will be used throughout the drawings to refer to the same or like parts.

FIG. 1a is a side view of a grasping tool in a closed configuration, according to an exemplary embodiment.

FIG. 1b is a side view of the grasping tool shown in FIG. 1a in an open configuration.

FIG. 2 is a side view of an end-effector arm, according to an exemplary embodiment.

FIG. 3 is a side view of an end-effector arm, according to another embodiment.

FIG. 4 is a side view of an end-effector arm, according to a further embodiment.

FIG. 5 is a side view of an end-effector arm, according to another embodiment.

FIG. 6 is a side view of an end-effector arm, according to a further embodiment.

FIG. 7 is a side view of an end-effector arm, according to an even further embodiment.

FIG. 8 is a side view of an end-effector arm, according to another embodiment.

FIG. 9 is a side view of an end-effector arm, according to another embodiment.

FIG. 10 is a side view of an end-effector arm, according to a further embodiment.

### DESCRIPTION OF THE EMBODIMENTS

Reference will now be made in detail to embodiments of the disclosure, examples of which are illustrated in the accompanying drawings.

### Overview

The present disclosure provides a tool including two arms forming a jaw-like structure. The tool may be an end-effector for holding/manipulating tissue and other target objects. The arms of the end-effector may be pivotally connected to one another to permit the arms to rotate between an open and a closed configuration. A proximal portion of one or both arms may be operably connected to a control member. The control member may extend through an elongated member, extending between the end-effector at its distal end to an end-effector actuating member located at its proximal end. Manipulating the actuating member may actuate the end-effector to effectuate opening and closing of the arms. The two arms may be differentiated as “upper” and “lower” jaws. Various configurations of end-effector actuation, structure, and functions are described in the embodiments of the disclosure. Further, as used in this disclosure, “distal” refers to a position or direction further from the user, “proximal” refers to a position or direction opposite “distal” and closer to the user, “medial” refers to a position or direction towards the centerline of the device, and “lateral” refers to a position or direction away from the centerline of the device.

More particularly, the present disclosure provides a device for securely holding and manipulating objects and/or tissues. Manipulation includes, but is not limited to, cutting, sectioning, stapling, clamping, cauterizing, grasping, holding, or scraping. The various methods of manipulation are described in detail hereinafter.

### Exemplary Embodiments

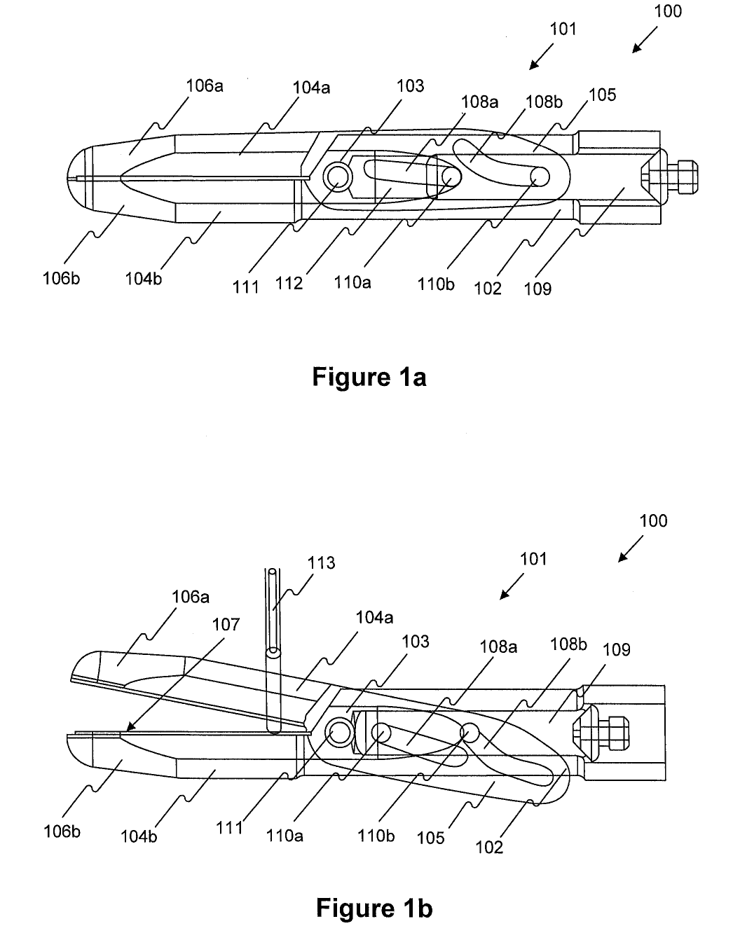

FIGS. 1a and 1b depict a grasping tool 100 in a closed and an open configuration, respectively, according to an embodiment of the present disclosure. The grasping tool 100 may include an end-effector 101 extending distally from an elongated member 102. The elongate member may define a slot 112 coincident with its longitudinal axis, and further define an opening at its proximal end. An actuating member 109 may be positioned inside the slot 112 and may be configured to translate movements from a controller or handle (not shown) at the proximal end of the elongated member 102. The elongate member 102 may include a clevis 103 that is located at its distal end.

The end-effector 101 may include two arms, an upper arm 104a and a lower arm 104b, (hereinafter, arms 104) pivotally connected to each other. The arms 104 may be pivotally coupled to the clevis 103, for example, via a pivot pin 111, to permit rotational movement between each other. The lower arm 104b may be integral with or fixedly connected to the elongate member 102 and/or clevis 103, and the upper arm 104a may include a proximal portion 105 located proximal to the clevis 103. The proximal portion 105 may define a distal slot 108a and a proximal slot 108b (hereinafter, slots 108). In other embodiments, the boundaries defining the slots 108 may be connected forming one continuous slot. The slot 112 may have an axis parallel to the elongated member 102, and the slot 112 also may define an opening at its proximal end. In addition, the arms 104 may include an upper distal portion 106a and a lower distal portion 106b (hereinafter, distal portions 106) located distally of the clevis 103. As shown in FIG. 1b, the distal portions 106 may include an inner surface 107 such as a surface configured to hold tissue or an object, such as a needle 113. In other embodiments (not shown), inner surface 107 may include a surface configured for cutting, sectioning, stapling, clamping, cauterizing, grasping, and/or scraping.

The actuating member 109 may include a distal pin 110a and a proximal pin 110b (hereinafter, pins 110) disposed longitudinally along its surface. The pins 110 may have a longitudital axis orthogonal to the axis of the actuating member 109 defining a circular cross section. In other embodiments (not shown), it is contemplated that the pins 110 may have other cross-sectional shapes such as polygonal (regular and irregular), elliptical, oval and other curved shapes. The pins 110 may be fixed to the actuating member 109, or the pins 110 may be configured to rotate with respect to the longitudinal axis of the pins 110. In other embodiments (not shown), the pins 110 may include a bearing such as a plastic or metal bushing or a ball bearing. The pins 110 may be slideably engageable with the slots 108, and the actuating member 109 may be slideably engageable with the slot 112, such that a linear displacement by the actuating member 109 within the slot 112 may cause the upper distal portion 106a to pivot with respect to the lower distal portion 106b. The lower arm 104b may be fixed with respect to the clevis 103, such that only the upper arm may be moveable. In another embodiment, the lower arm 104b may also includes slots (not shown) slideably engageable with the pins 110, such that both arms may be moveable with respect to the elongate member 102.

As shown in FIGS. 1a and 1b, each one of the pins 110 is located within one of the slots 108. It has been contemplated in other embodiments (not shown) that more than one pin may be located within the same slot, such as two or three pins, for example. Furthermore, multiple slots may include a plurality of pins, such as two pins in a first slot, and three pins in a second slot, etc. In other embodiments (not shown) the pins 110 and the slots 108 may be reversed, such that slots are formed within the actuating member 109 and engaged by fixed pins located on the proximal portions 105 of the one of more arms 104.

The distal slot 108a may have a different configuration or profile than slot 108b to allow for each of the pins 110 to simultaneously transfer a force from the actuating member 109 to the arms 104 while being actuated. Each of the pins 110 may be configured to simultaneously transfer a force throughout the entire range of motion, or each of the pins 110 may be configured to simultaneously transfer a force throughout a portion of the range of motion. Further, each of the pins 110 may be configured to transfer an equal amount of force throughout a range of motion, or each of the pins 110 may be configured to transfer an unequal amount of force throughout a range of motion.

Each of the pins 110 may be comprised of the same material or a different material. At least one of the pins 110 may be comprised of a deformable material to compensate for manufacturing tolerances in the slots 108. At least one of the pins 110 may be comprised of a lubricious material to reduce wear, improve longevity, and compensate for manufacturing tolerances in the slots 108. Further, one of pins 110 may be comprised of a metallic material or a plastic material such as PTFE or ePTFE.

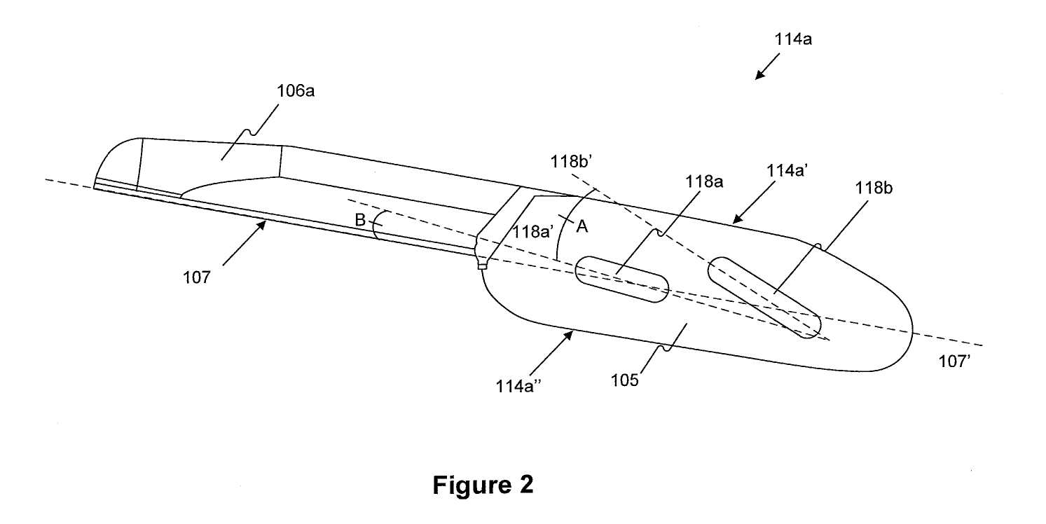

In an embodiment shown in FIG. 2, an arm 114a may define a distal slot 118a and a proximal slot 118b (hereinafter, slots 118). Slots 118 may each have a generally linear longitudinal axis. The axis of slot 118a may form an angle A with respect to the axis of slot 118b. Angle A may include rays 118a′ and 118b′ each coincident with the respective axes of slots 118a and 118b. The vertex of angle A may be located proximally to the center of slot 118b (center of slot 118b is not labeled) with ray 118b′ intersecting the lateral edge 114a′ of arm 114a. Additionally, ray 118a′ also may intersect the lateral edge 114a′ of arm 114a. The length of slot 118a′ may be shorter than the length of slot 118b′. Inner surface 107 may define an axis 107′ coincident with its inner edge, and ray 118a′ may form an angle B with axis 107′. Also, the vertex of angle A may be located medially to the axis 107′. In addition, angle B may be smaller than angle A.

In other embodiments, the vertex of angle A may be located distally to the center of slot 118a (center of slot 118a is not labeled) or the vertex of angle A may be generally located between the centers of slots 118. The vertex of angle A may be coincident with or lateral to axis 107′. Either one, all, or none of rays 118a′ and 118b′ may intersect the lateral edge 114a′ of 114a, while one, all, or none of the remaining rays 118a′ and 118b′ may intersect the medial edge 114b′ of 114a. Either one or both of rays 118a′ and 118b′ may by parallel to axis 107′. In addition, the length of slot 118a may be longer or equal to the length of slot 118b. Additionally, angle B may be equal to or greater than angle A.

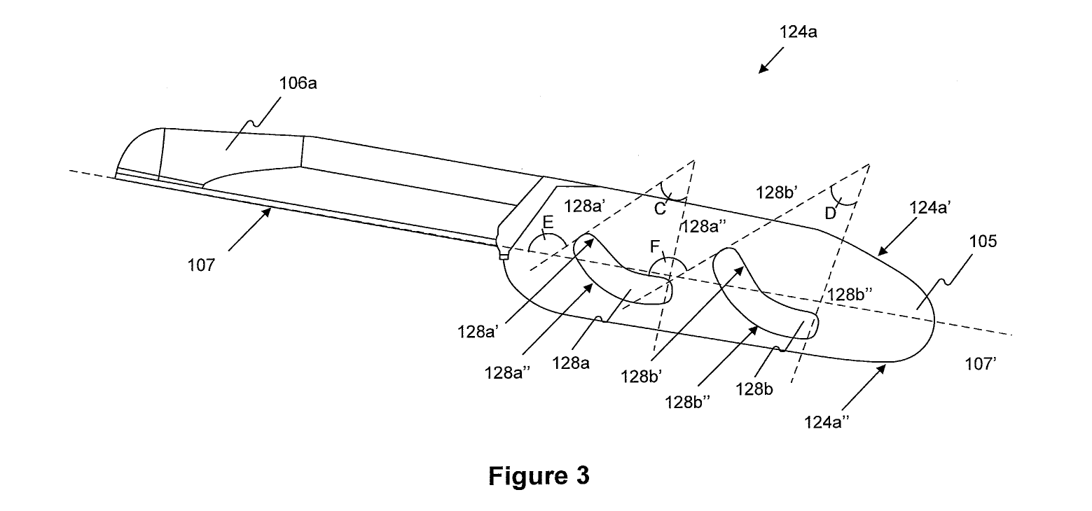

In another embodiment shown in FIG. 3, an arm 124a may define a distal slot 128a and a proximal slot 128b (hereinafter, slots 128). Slots 128 may each have a generally curvilinear longitudinal axis. The length of the slot 128a may be shorter than the length of the slot 128b. Each of slots 128 may have lateral edges 129a′ and 129b′ (hereinafter, lateral edges 129′) and medial edges 129a″ and 129b″ (hereinafter, medial edges 129″) that are generally closer to the respective lateral edge 124a′ and medial edge 124a″ of arm 124a. Each of slots 128 may have a constant curvature with its medial and lateral edges 129″ and 129′ having the same radius of curvature. Each of the slots 128 may be convexo-concave with concave lateral edges 129′. Each of the lateral edges 129a′ and 129b′ of slots 128a and 128b may be defined by an arc having angles C and D, respectively. Each of angles C and D may include generally distal rays 128a′ and 128b′, respectively, and generally proximal rays 128a″ and 128b″, respectively. The vertices of angles C and D may be generally located closer to the lateral edge 124a′ of arm 124a than the medial edge 124a″ of arm 124a. Each of the slots 128 may have a center (not labeled) located at equidistance from the edges of each slot along the longitudinal axis from an end of the slot. The center of each of the slots 128 may be located medially to the axis 107′. The intersection of rays 128a′ and 128b′ with axis 107′ may form angles E and F respectively. Angle E may be greater than 90 degrees, while angle F may be greater than angle E. Rays 128a′, 128a″, 128b′, and 128b″ may each intersect the medial edge 124″ of arm 124a.

In other embodiments, either one or both of angles C and D may be generally located closer to the medial edge 124a″ of arm 124a than the lateral edge 124a′ of arm 124a. The center of one or both of the slots 128 may be located laterally to or coincident with the axis 107′. Angle E may be 90 degrees or less, and angle F may be equal to or less than angle E. One or more of rays 128a′, 128a″, 128b′, and 128b″ may each intersect the lateral edge 124a′ of arm 124a and/or one or more of rays 128a′, 128a″, 128b′, and 128b″ may be parallel to axis 107′. Each of the slots 128 may have a constant curvature with its medial and lateral edges 129″ and 129′ having different radii of curvature. For example, the medial edges 129″ of slots 128 may have a larger or smaller radius of curvature than the lateral edges 129′ of slots 128. Further, the radius of curvature for the medial and lateral edges 129″ and 129′ of each of the slots 128 may or may not be concentric to each other. Each of the slots 128 may be convexo-concave with concave medial edges 129″. Each of the slots 128 may have medial and lateral edges 128″ and 128′ that are both either convex or concave. In addition, slots 128 may have a curvature that is not constant (multiple radii of curvature) with each respective arc of curvature forming an angle similar to angles C and D as discussed above. In the case where a slot has multiple arcs of curvature, two or more of the vertices may be closer to the medial edge 124a″ of arm 124a or the lateral edge 124a′ of arm 124a. Alternatively, at least two of the vertices may be closer to opposing edges of the arm 124a. Further, one or both of the lateral and medial edges 128′ and 128″ of the slots 128 may include both convex and concave portions.

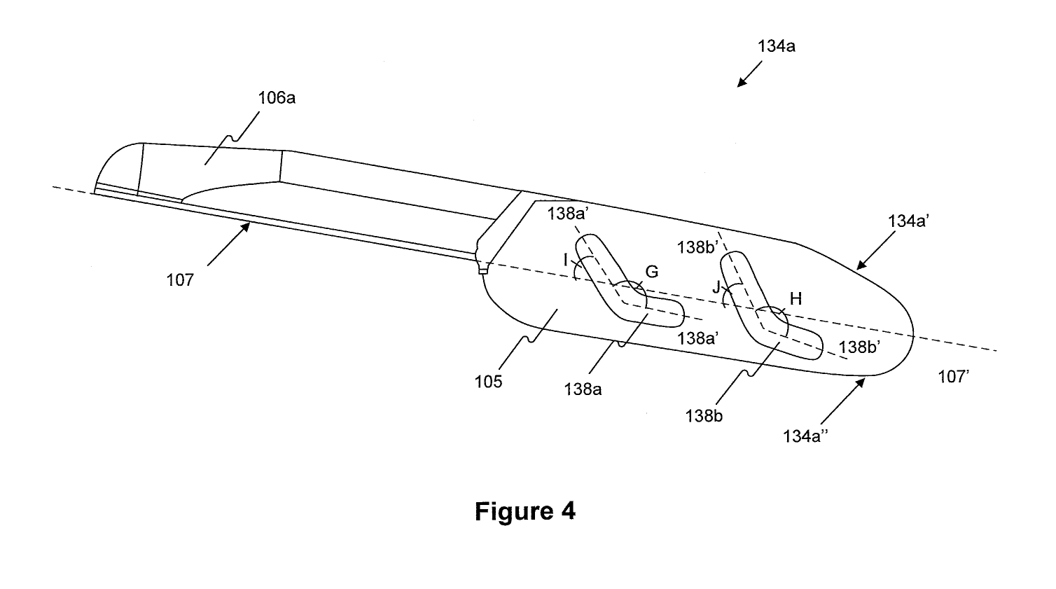

In another embodiment shown in FIG. 4, an arm 134a may define a distal slot 138a and a proximal slot 138b (hereinafter, slots 138). Slot 138a may include two generally linear segments combined to form an angle G having rays 138a′ and 138a″ coincident with each of the linear segments. Further, slot 138b may include two generally linear segments combined to form an angle H having rays 138b′ and 138b″ coincident with each of the linear segments. The length of the linear segment corresponding to ray 138a′ may be shorter than the length of the linear segment corresponding to ray 138b′ and longer than the linear segment corresponding to ray 138a″, while the length of the linear segment corresponding to ray 138a″ may be shorter than the length of the linear segment corresponding to ray 138b″. Additionally, the length of the linear segment corresponding to ray 138b″ may be shorter than the length of the linear segment corresponding to ray 138b′. Angles G and H may be substantially equal and the vertex of each of the angles G and H may be located medially to axis 107′. Angle I may be less than 90 degrees, and ray 138a′ may intersect the lateral edge 134a′ of arm 134a. Angle J may be greater than angle I, and ray 138b′ may also intersect the lateral edge of arm 134a. Additionally, angles G and H may face the lateral edge 134a′ of arm 134a.

In other embodiments, either one or both of the vertices of angles G and H may be located laterally to or coincident with the axis 107′. Angle I may be 90 degrees or more, and angle G may be greater than or less than angle H. The length of the linear segment corresponding to ray 138a′ may be longer than or equal to the length of the linear segment corresponding to ray 138b′, while the length of the linear segment corresponding to ray 138a″ may be longer than or equal to the length of the linear segment corresponding to ray 138b″. Ray 138a′ may intersect the medial edge of arm 134a and/or may be parallel with axis 107′. Angle J may be less than or equal to angle I, and ray 138b′ may also intersect the medial edge 134a″ of arm 134a. Additionally, angles G and H may face the medial edge 134a″ of arm 134a. In other embodiments, slots having three or more linear segments may exist with each segment and each angle formed between adjacent segments being similar to the segments and angles of slots 138 described above.

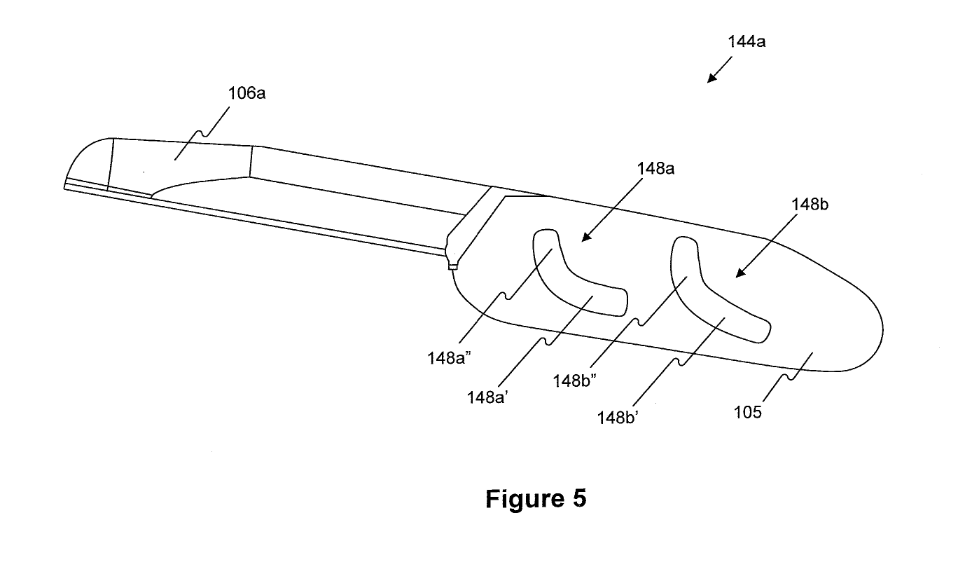

In another embodiment shown in FIG. 5, an arm 144a may define a distal slot 148a and a proximal slot 148b (hereinafter, slots 148). Slot 148a may have a profile including a generally linear segment 148a′ joined with a curvilinear segment 148a″. Further, slot 148b may have a profile including a generally linear segment 148b′ joined with a curvilinear segment 148b″. The linear segments 148a′ and 148b′ may be similar to linear slots 118 as described above and the curvilinear segments 148a″ and 148b″ may be similar to curvilinear slots 128 as described above.

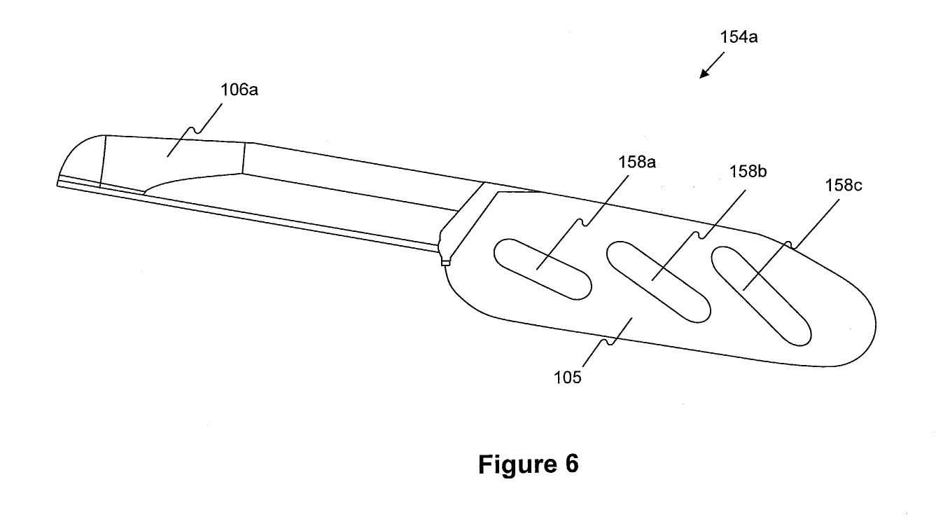

In another embodiment shown in FIG. 6, an arm 154a may define a distal slot 158a, a central slot 158b, and a proximal slot 158c (hereinafter, slots 158). Slots 158 may each have generally linear longitudinal axis. The axis of slot 158a may form an angle with respect to the axis of slot 158b, and a larger angle with respect to slot 158c. The length of the slot 158b may be shorter than the length of the slot 158c and larger than the length of the slot 158a. Furthermore, each of slots 158 may be similar to slots 118 as described above.

In other embodiments, an arm may include any combination of linear, curvilinear, linear-segmented, and linear-curvilinear hybrid slots similar to slots 118, 128, 138, and 148, respectively, as disclosed herein. Additionally, an arm may include three or more slots of any geometry disclosed herein.

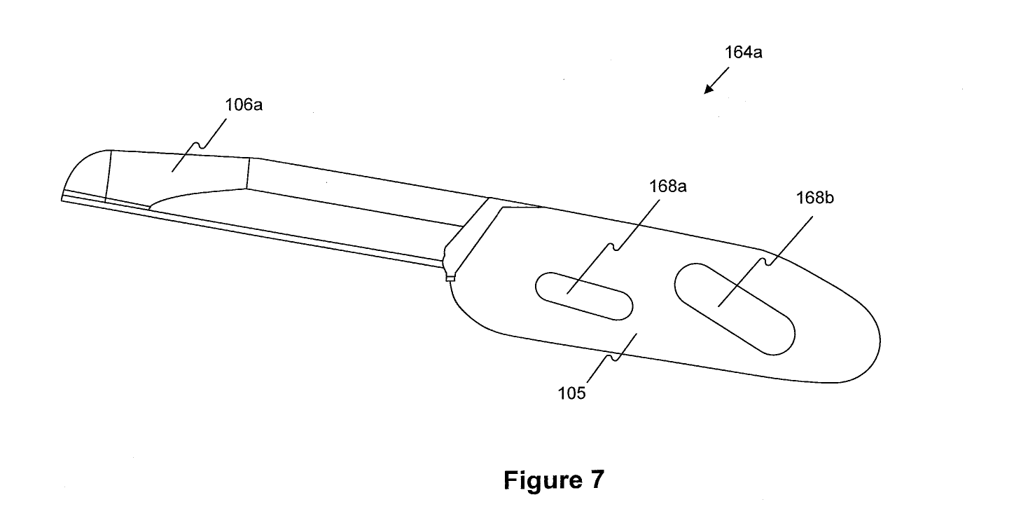

In another embodiment shown in FIG. 7, an arm 164a may define a distal slot 168a and a proximal slot 168b (hereinafter, slots 168). Slots 168 may be similar to slots 118 as described above. In addition, the width of slot 168b may be wider than slot 168a to allow for manufacturing tolerances.

In other embodiments, slot 168a may be wider than slot 168b. Additionally, the medial and lateral sides of one or both of slots 168 may be wider at a proximal end and narrow towards a distal end as to form an angle between the sides. Alternatively, the medial and lateral sides of one or both of slots 168 may be wider at a distal end and narrow towards a proximal end. The inner surfaces of the slots 168 may be substantially smooth or rough. It is contemplated that any of the other embodiments disclosed herein may include one or more slots with unequal widths.

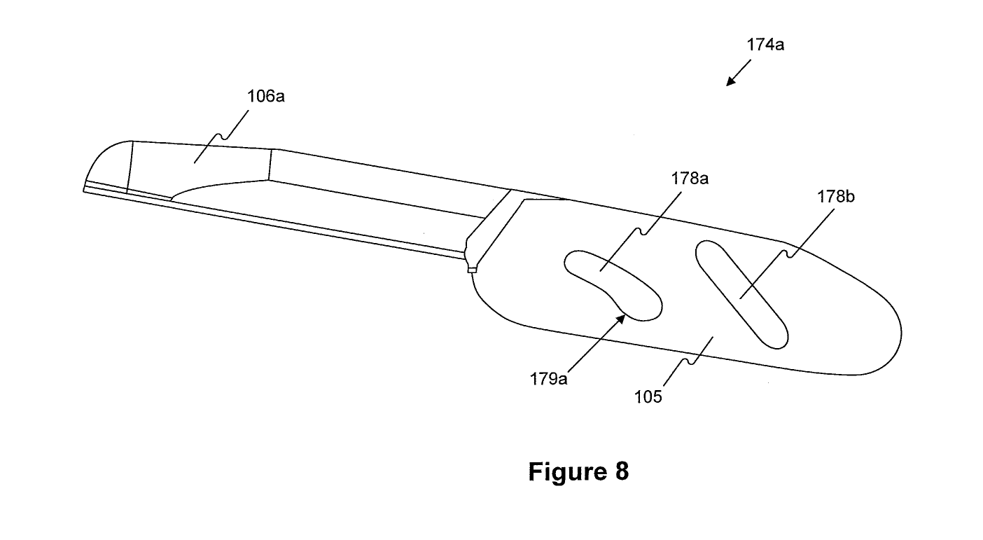

In another embodiment shown in FIG. 8, an arm 174a may define a distal slot 178a and a proximal slot 178b (hereinafter, slots 178). Slots 178 may be similar to slots 118 as described above. In addition, the width of slot 178a may be unequal along its axis, such as having a recess of a larger width on its medial side towards its proximal end. In this exemplary configuration, the wide portion of the slot 178a may behave as a clutch to engage/disengage the pin 110a near the closed position for a pair of jaws (not shown).

In other embodiments, the wide portion of slot 178a may be located on its medial side towards its distal end. In this exemplary configuration, the wide portion of the slot 178a may behave as a clutch to engage/disengage the pin 110a near the open position for a pair of jaws (not shown). Slot 178 may also include wide portion on its lateral side at either its proximal or distal end. Also, the slot 178b also may include a wide portion on either its medial or lateral side towards its proximal or distal ends to engage/disengage the pin 110b near the open or closed position for a pair of jaws (not shown). Additionally, one or both of the slots 178 may include a wide portion at any position between the distal and proximal ends on either its medial or lateral sides. One or both of the slots 178 may also include a wide portion at both of its ends forming a “bone” or “dumbbell” shape to engage/disengage the pin 110b near both the open and closed positions for a pair of jaws (not shown). It is also contemplated that any of the embodiments disclosed herein may include one or more slots with one or more wide portions on a lateral and/or medial side and at a proximal and/or distal end thereof.

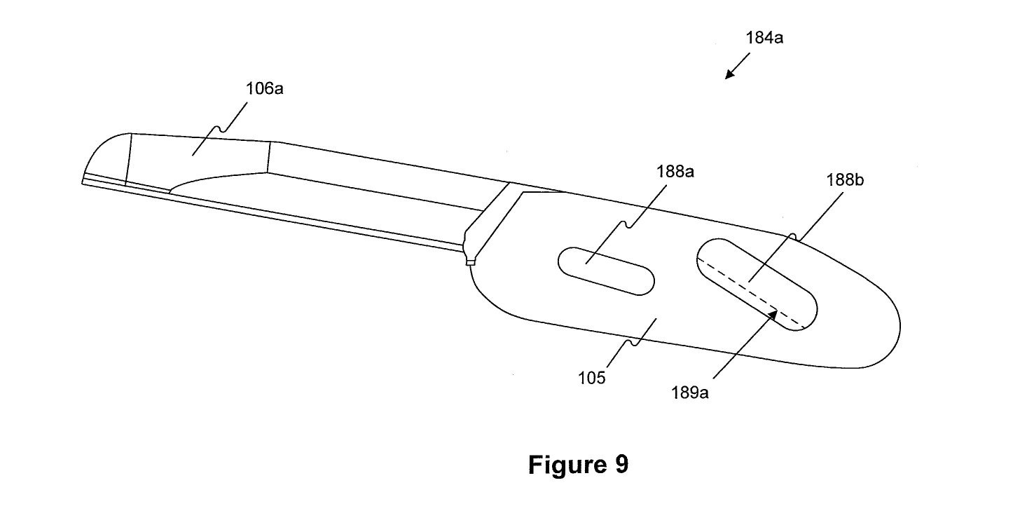

In another embodiment shown in FIG. 9, an arm 184a may define a distal slot 188a and a proximal slot 188b (hereinafter, slots 188). Slots 188 may be similar to slots 118 as described above. In addition, a portion of the proximal portion 105 of the arm 184a may include a shape memory alloy (SMA) defining a moveable edge 189b. The moveable edge 189b may be configured to change the width of the slot 188b by changing the temperature of the SMA near the movable edge 189b. Temperature may be changed by the conversion of electrical energy to thermal energy and may be controlled by, for example, a heating element (not shown) or a peltier heater (not shown). In this exemplary configuration, the moveable edge 189b may behave as a clutch to engage/disengage the pin 110b. When the moveable edge 189b is in the wide position, the pin 110b may be disengaged from the slot 188b and when the moveable edge is in the narrow position, the pin 110 may be engaged with the slot 188b.

In other embodiments slot 188a may include a moveable edge to engage/disengage the pin 110a with the slot 188a. Also, both slots 188 may include a moveable edge and each moveable edge may be actuated independently from each other. The moveable edge may be located on a lateral surface, medial surface, or both surfaces of the slots. Further, the proximal end, distal end, or both surfaces of slots 188 may include moveable edges and may be actuated to shorten or lengthen the slots 188. It is also contemplated that any of the embodiments disclosed herein may include a SMA and in addition one or more slots with a moveable edge.

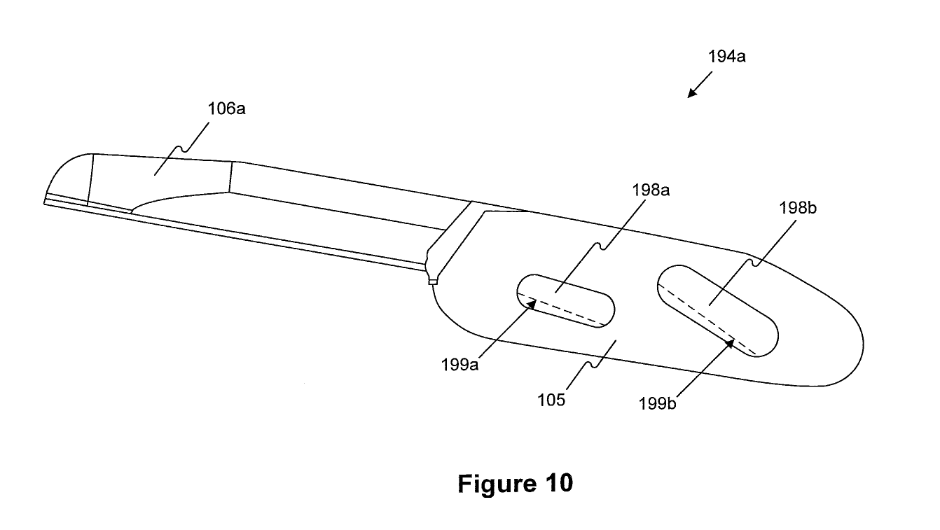

In another embodiment shown in FIG. 10, an arm 194a may define a distal slot 198a and a proximal slot 198b (hereinafter, slots 198). Slots 198 may be similar to slots 118 as described above. In addition, a portion of the proximal portion 105 of the arm 194a may include a SMA defining moveable edges 199a and 199b (hereinafter, moveable edges 199). The moveable edges 199 may be configured to non-uniformly change the width of the slots 198 by changing the temperature of the SMA near the edges 199. Temperature may be changed by the conversion of electrical energy to thermal energy and may be controlled by, for example, a heating element (not shown) or a peltier heater (not shown). In this exemplary configuration, the moveable edges 199 may behave as both a clutch and a gear to engage/disengage the pins 110 and change the torque profile of an end-effector (not shown). When the moveable edges 199 become narrow towards their lateral edges, torque may increase. In addition, when the moveable edges become narrow towards their medial edges, torque may decrease. Each of the moveable edges 199 may be controlled independently of each other or the actuation of both of the edges 199 may be coupled together.

In other embodiments, the moveable edges 199 may change shape during actuation, such that a curvilinear shape results. It is also contemplated that any of the embodiments disclosed herein may include a SMA and in addition one or more slots with a moveable edge.

In other embodiments similar to the arms 184a and 185a, movement of the moveable edges 189 and 199 may be actuated mechanically, by a controller and linkage system (not shown). Also, the pins 110 in any of the embodiments disclosed herein may be mechanical actuated by a controller and linkage system (not shown) to selectively engage either of the pins 110.

It is contemplated that the arms, slots, and pins may have electrically conductive surfaces, electrically insulating surfaces, or combinations thereof. For example, it may be desired to transfer electrosurgical energy to a target tissue through an end-effector. A conductive pathway may exist from an external power source (not shown), through conductive wiring (not shown), through a pin, through an inner surface of a slot, through an arm, and to an inner surface. In other embodiments, a portion of a slot may have an electrically insulating portion and an electrically conductive portion. This may allow, for example, an end-effector to transfer electrosurgical energy to a target tissue while the arms are in the open position, and to prevent any electrical energy to be transferred while the arms are in the closed position. It is contemplated that any portion of a slot, including all of the slot or none of the slot, may be electrically conductive such that an end-effector may be electrically conductive over a predetermined range of motion.

Other embodiments of the present disclosure will be apparent to those skilled in the art from consideration of the specification and practice of the embodiments disclosed herein. It is intended that the specification and examples be considered as exemplary only, with a true scope and spirit of the invention being indicated by the following claims.

What is claimed is:

1. A medical device comprising:
an elongate member having a proximal end, a distal end, and a lumen extending therebetween;
an end-effector disposed at the distal end of the elongate member, the end-effector including a first arm and a second arm;
the first arm defining a first slot and a second slot;
the medical device further including an actuating member, the actuating member including a plurality pins; and
wherein each of the pins are slideably engageable with the first and second slots.

2. The medical device of claim 1, wherein the first slot is proximal to the second slot.

3. The medical device of claim 1, wherein at least one of the first and second slots defines a curvilinear path.

4. The medical device of claim 1, wherein at least one of the first and second slots defines a linear path.

5. The medical device of claim 1, wherein at least one of the plurality of pins is selectively engageable.

6. The medical device of claim 1, wherein at least one of the plurality of pins includes a deformable material.

7. The medical device of claim 1, wherein the plurality of pins includes a first pin and a second pin;
wherein the first and second pins are comprised of different materials.

8. The medical device of claim 1, wherein the second arm is fixed with respect to the elongate member.

9. A medical device comprising:
an elongate member having a proximal end, a distal end, and a lumen extending therebetween;
an end-effector disposed at the distal end of the elongate member, the end-effector including a first arm and a second arm;
the medical device further including an actuating member, the actuating member including a plurality of pins;
wherein each of the pins are configured to transfer an applied force to the first arm; and
wherein the first arm is configured to rotate with respect to the second arm upon receiving the applied force.

10. The medical device of claim 9, wherein the first arm defines a first slot and a second slot, wherein the first slot is located distally to the second slot.

11. The medical device of claim 9, at least one of the first and second slots defines a curvilinear path.

12. The medical device of claim 9, wherein at least one of the plurality of pins is selectively engageable.

13. The medical device of claim 9, wherein at least one of the plurality of pins includes a deformable material.

14. The medical device of claim 9, wherein the second arm is fixed with respect to the elongate member.

15. A medical device comprising:
an elongate member having a proximal end, a distal end, and a lumen extending therebetween;
an end-effector disposed at the distal end of the elongate member, the end-effector including a first arm and a second arm;
the first arm defining a first slot and a second slot, the first slot and the second slot having a different profile from each other;
the medical device including an actuating member configured to transfer a plurality of applied forces to the first and second slots of the first arm; and
wherein the first arm is configured to rotate with respect to the second arm upon receiving the applied force.

16. The medical device of claim 15, wherein at least one of the first and second slots define a curvilinear path.

17. The medical device of claim 15, wherein at least one of the first and second slots define a linear path.

18. The medical device of claim 15, wherein the actuating member is configured to transfer the plurality of applied forces to the first slot.

19. The medical device of claim 15, wherein the plurality of applied forces includes a first force and a second force;
wherein the first force is configured to be applied to the first slot and the second force is configured to be applied to the second slot; and
wherein the first force is different from the second force.

20. The medical device of claim 15, wherein the second arm is fixed with respect to the elongate member.
